(19) United States
(12) Patent Application Publication
(10) Pub. No.: US 2017/0314002 A1
(43) Pub. Date: Nov. 2, 2017

(54) DIMERIC PROTEINS FOR SPECIFIC TARGETING OF NUCLEIC ACID SEQUENCES

(71) Applicant: Bio-Rad Laboratories, Inc., Hercules, CA (US)

(72) Inventor: Xiaosong GONG, Richmond, CA (US)

(21) Appl. No.: 15/499,564

(22) Filed: Apr. 27, 2017

Related U.S. Application Data
(60) Provisional application No. 62/329,740, filed on Apr. 29, 2016.

(51) Int. Cl. C12N 9/22; C12N 15/11; C12N 15/62; C12N 2310/20

(52) U.S. Cl. CPC C12N 9/22; C12N 15/62; C12N 15/11; C12N 2310/20

(57) ABSTRACT

Polypeptide constructs comprising a first sequence-specific DNA-binding protein linked to a second sequence-specific DNA-binding protein and methods for using the polypeptide constructs are provided.

### CROSS-REFERENCE TO RELATED PATENT APPLICATIONS

The present application claims benefit of priority to U.S. Provisional Patent Application No. 62/329,740, filed Apr. 29, 2016, which is incorporated by reference.

### BACKGROUND OF THE INVENTION

“Cas9” or “Cas9 nuclease” refers to an RNA-guided nuclease comprising a Cas9 protein. Catalytically-active Cas9 comprises an active DNA cleavage domain of Cas9 and a guide RNA (gRNA) binding domain. A Cas9 nuclease is also referred to sometimes as a casn1 nuclease or a CRISPR (clustered regularly interspaced short palindromic repeat)-associated nuclease.

CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNA. However, single guide RNAs can be engineered so as to incorporate aspects of both the crRNA and tracrRNA into a single RNA species. See e.g., Jinek M., Chylinski K., Fonfara I., Hauer M., Doudna J. A., Charpentier E. Science 337:816-821 (2012). Cas9 recognizes a short motif in the CRISPR repeat sequences (the PAM or protospacer adjacent motif) to help distinguish self versus non-self. Cas9 nuclease sequences and structures have been described (see, e.g., Ferretti et al., Proc. Natl. Acad. Sci. U.S.A. 98:4658-4663 (2001); Mojica et al., Microbiology 155: 733-740 (2009); Deltcheva E., et al., Nature 471:602-607 (2011); and Jinek M., et al. Science 337:816-821 (2012). Cas9 orthologs have been described in various species, including, but not limited to, S. pyogenes and S. thermophilus.

Whether CRISPR is used for genome editing, genome targeting, or other uses, it is generally desirable that the CRISPR system specifically target nucleotide sequences of interest and avoid non-target (“off-target”) sequences in the genome. For example, Wang et al., Nature Biotechnology 33:175-178 (2015) showed that off-target frequencies can be high. In this paper, the off-target integration was 88% of the total integration sites for the WAS CR-4 target. The on-target integration was only 12%.

Argonaute (Ago) proteins can also be used to target polynucleotide sequences. For example, Ago proteins are ubiquitously expressed and bind to siRNAs or miRNAs to guide post-transcriptional gene silencing either by destabilization of the mRNA or by translational repression. Ago proteins generally have at least four domains: an N-terminal domain, a PAZ domain, a Mid domain and a C-terminal PIWI domain. See, e.g., Hutvagner, Nature Reviews Molecular Cell Biology 9 (1): 22-32 (2008) and Meister, Nature Reviews Genetics 14:447-459 (2013).

### Definitions

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and nucleic acid chemistry and hybridization described below are those well-known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well-known and commonly employed in the art.

As used herein, “nucleic acid” means DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, points of attachment and functionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications such as capping with a fluorophore (e.g., quantum dot) or another moiety.

The terms “oligonucleotide” or “polynucleotide” or “nucleic acid” interchangeably refer to a polymer of monomers that can be corresponded to a ribose nucleic acid (RNA) or deoxyribose nucleic acid (DNA) polymer, or analog thereof. This includes polymers of nucleotides such as RNA and DNA, as well as modified forms thereof, peptide nucleic acids (PNAs), locked nucleic acids (LNA™), and the like. In certain applications, the nucleic acid can be a polymer that includes multiple monomer types, e.g., both RNA and DNA subunits.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, .gamma.-carboxyglutamate, and O-phosphoserine. Amino acid analogs refers to compounds that have the same basic chemical structure as a naturally occurring amino acid, i.e., a carbon atom that is bound to a hydrogen atom, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The term “encoding” refers to a polynucleotide sequence encoding one or more amino acids. The term does not require a start or stop codon. An amino acid sequence can be encoded in any one of six different reading frames provided by a polynucleotide sequence.

The term “promoter” refers to regions or sequence located upstream and/or downstream from the start of transcription and which are involved in recognition and binding of RNA polymerase and other proteins to initiate transcription.

A “vector” refers to a polynucleotide, which when independent of the host chromosome, is capable replication in a host organism. Preferred vectors include plasmids and typically have an origin of replication. Vectors can comprise, e.g., transcription and translation terminators, transcription and translation initiation sequences, and promoters useful for regulation of the expression of the particular nucleic acid. Any of the polynucleotides described herein can be included in a vector.

Sequences are “substantially identical” to each other if they have a specified percentage of nucleotides or amino acid residues that are the same (e.g., at least 60%, 65%, 70%, 75%, 80%, 85%, 90%, 91%, 92%, 93%, 94%, 95%, 96%, 97%, 98%, or 99% identity over a specified region), when compared and aligned for maximum correspondence over a comparison window, or designated region as measured using one of the following sequence comparison algorithms or by manual alignment and visual inspection.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Accelrys), or by manual alignment and visual inspection.

Algorithms suitable for determining percent sequence identity and sequence similarity are the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a word length (W) of 11, the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression cassette” refers to a nucleic acid construct that, when introduced into a host cell, results in transcription and/or translation of an RNA or polypeptide, respectively.

### BRIEF SUMMARY OF THE INVENTION

Provided herein is a polypeptide construct comprising a first sequence-specific DNA binding protein, selected from a Cas9 protein or an Ago protein, linked to a second sequence-specific DNA binding protein.

In some embodiments, the first sequence-specific DNA binding protein is a Cas9 protein. In some embodiments, the Cas9 protein is a dCas9 protein. In some embodiments, the Cas9 protein is a nuclease-active (i.e., “active”) Cas9 protein. In some embodiments, a first dCas9 polypeptide is linked to a second Cas9 polypeptide. In some embodiments, the second Cas9 polypeptide is a second dCas9 polypeptide. In some embodiments, the second Cas9 polypeptide is a nuclease-active Cas9 polypeptide. Cas9 proteins can be naturally-occurring or variants thereof that retain at least DNA-binding activity. In some embodiments, the Cas9 proteins are substantially identical to a naturally-occurring Cas9 protein.

In some embodiments, the first sequence-specific DNA binding protein is covalently linked to the second sequence-specific DNA binding protein. In some embodiments, the first sequence-specific DNA binding protein is covalently linked to the second sequence-specific DNA binding protein as a translational fusion protein. In some embodiments, the first sequence-specific DNA binding protein is linked to the second sequence-specific DNA binding protein via a dimerization domain(s).

In some embodiments, the first dCas9 polypeptide is bound to a first guide RNA and the second Cas9 polypeptide is bound to a second guide RNA and the first dCas9 polypeptide is linked to the second Cas9 polypeptide via an interaction of the first guide RNA to the second guide RNA. In some embodiments, the first dCas9 polypeptide and the second Cas9 polypeptide are bound to an RNA, said RNA comprising a first Cas9 guide sequence; a first tracr RNA sequence; a second Cas9 guide sequence; and a second tracr RNA sequence, wherein: the first dCas polypeptide binds to the first tracr RNA sequence and the second Cas9 polypeptide binds to the second tracr sequence; or the first dCas polypeptide binds to the second tracr RNA sequence and the second Cas9 polypeptide binds to the first tracr sequence, such that the first dCas polypeptide and the second Cas polypeptide are bound together via the RNA.

In some embodiments, the second sequence-specific DNA binding protein comprises a TALE DNA binding domain or a zinc finger protein.

In some embodiments, the first sequence-specific DNA binding protein is an Ago protein. In some embodiments, the second sequence-specific DNA binding protein comprises a Cas9 protein, a TALE DNA-binding domain, or a zinc finger protein.

Also provided is a cell comprising the polypeptide construct as described above or elsewhere herein. In some embodiments, the cell is a eukaryotic cell. In some embodiments, the cell is a human cell.

Also provided is a nucleic acid encoding the translational fusion protein as described above or elsewhere herein. Also provided is a cell comprising the nucleic acid. In some embodiments, the cell is a eukaryotic cell. In some embodiments, the cell is a human cell.

Also provided is a method of introducing the polypeptide construct into a eukaryotic cell, the method comprising, introducing the polypeptide construct into the cell. In some embodiments, the polypeptide construct is introduced into the cell by electroporation. In some embodiments, the first sequence-specific DNA binding protein is covalently linked to the second sequence-specific DNA binding protein as a translational fusion protein, and a nucleic acid encoding the translational fusion protein is introduced into the cell, wherein the polypeptide construct is expressed in the cell, thereby introducing the polypeptide construct into the cell.

Also provided is an RNA comprising from 5′ to 3′: a first Cas9 guide sequence; a first tracr RNA sequence; a second Cas9 guide sequence; and a second tracr RNA sequence.

Also provided is an RNA comprising from 5′ to 3′: a first Cas9 guide sequence; a second Cas9 guide sequence; and a first tracr RNA sequence followed by a second tracr RNA sequence; or a second tracr RNA sequence followed by a first tracr RNA sequence.

Also provided is an expression cassette comprising a promoter operably linked to a polynucleotide encoding the RNA as described above. Also provided is a cell comprising the expression cassette. In some embodiments, the cell is a eukaryotic cell. In some embodiments, the cell is a human cell.

Also provided is a composition comprising: the RNA as described above; a first dCas9 polypeptide; and a second Cas9 polypeptide. In some embodiments, the second Cas9 polypeptide is a second dCas9 polypeptide. In some embodiments, the second Cas9 polypeptide is a nuclease-active Cas9 polypeptide. Also provided is a cell comprising the composition. In some embodiments, the cell is a eukaryotic cell. In some embodiments, the cell is a human cell.

Also provided is a method of introducing the composition into a cell. In some embodiments, the method comprises introducing the composition, or all of the components thereof (the RNA, the first dCas9 polypeptide and the second Cas9 polypeptide), into the cell. In some embodiments, the method comprises introducing the Cas9 or dCas9/guide RNA/tracrRNA complex, which has active binding activity to the target sequence, into the cell.

### BRIEF DESCRIPTION OF THE DRAWINGS

FIG. 1 schematically displays two embodiments for linking two Cas9 polypeptides together.

FIG. 2 schematically displays two alternative embodiments for linking two Cas9 polypeptides together.

### DETAILED DESCRIPTION OF THE INVENTION

### Introduction

Provided herein are polypeptide constructs comprising a first sequence-specific DNA binding protein (e.g., selected from a Cas9 or Ago protein) linked to a second sequence-specific DNA binding protein. The terms “first” and “second” when used with reference to polypeptides is simply to more clearly distinguish the two polypeptide sequences and is not intended to indicate order. In some embodiments, the second sequence-specific DNA binding protein is selected from a Cas9 polypeptide, an Ago polypeptide, a TALE DNA-binding domain, or a zinc finger.

In some embodiments the first sequence-specific DNA binding protein is a Cas9 protein. The Cas9 protein can be a catalytically dead Cas9 (“dCas9”) polypeptide or an active Cas9 polypeptide. In some embodiments, the polypeptide construct comprises a catalytically dead Cas9 (“dCas9”) polypeptide or an active Cas9 polypeptide linked to a second Cas9 polypeptide. The second Cas9 polypeptide can be active or catalytically dead as desired.

The described polypeptide constructs are expected to have improved specificity (reduced “off-target activity) compared to the second Cas9 polypeptide alone. The increased specificity should result when respective guide RNA sequences target adjacent genomic sequences such that both the first dCas9 polypeptide and the second Cas9 polypeptide bind sequences in close proximity. The resulting double binding event (i.e., both the first dCas9 polypeptide and the second Cas9 polypeptide target their respective sequences in the genome) will greatly decrease off-target binding.

The described polypeptide constructs can be applied for any use of a Cas9 or dCas9 polypeptide, including but not limited to genome editing (with an active Cas9) or targeting of genomic regions for other purposes (e.g., with dCas9). See, e.g., Maeder, et al., Nature Methods 10:977-979 (2013); Gilbert, et al., Cell 154(2): 442-451 (2013); Hu et al., Nucleic Acids Research 42(7): 4375-90 (2014).

The first dCas9 polypeptide can be any Cas9 polypeptide lacking catalytic activity. The dCas9 polypeptide can be from any species, for example, but not limited to from S. pyogenes. Methods for generating a Cas9 protein (or a fragment thereof) having an inactive DNA cleavage domain are known. See, e.g., Jinek et al., Science 337:816-821 (2012); Qi et al., Cell. 28; 152(5):1173-83 (2013). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. In some embodiments, the dCas9 polypeptide contains mutations in the D10 and H840 residues, e.g., D10N or D10A and H840A or H840N or H840Y, to render the nuclease portion of the protein catalytically inactivated Cas9. For example, the mutations D10A and H840A completely inactivate the nuclease activity of S. pyogenes Cas9. An exemplary dCas9 sequence (D10A and H840A) is as follows:

(SEQ ID NO: 1)
MDKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFF
HRLEESFLVEEDKKHERHPIFGNIVDEVAYHEKYPTIYHLRKKLVDSTD
KADLRLIYLALAHMIKFRGHFLIEGDLNPDNSDVDKLFIQLVQTYNQLF
EENPINASGVDAKAILSARLSKSRRLENLIAQLPGEKKNGLFGNLIALS
LGLTPNFKSNFDLAEDAKLQLSKDTYDDDLDNLLAQIGDQYADLFLAAK
NLSDAILLSDILRVNTEITKAPLSASMIKRYDEHHQDLTLLKALVRQQL
PEKYKEIFFDQSKNGYAGYIDGGASQEEFYKFIKPILEKMDGTEELLVK
LNREDLLRKQRTFDNGSIPHQIHLGELHAILRRQEDFYPFLKDNREKIE
KILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFN
ASLGTYHDLLKIIKDKDFLDNEENEDILEDIVLTLTLFEDREMIEERLK
TYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKK
GILQTVKVVDELVKVMGRHKPENIVIEMARENQTTQKGQKNSRERMKRI
EEGIKELGSQILKEHPVENTQLQNEKLYLYYLQNGRDMYVDQELDINRL
SDYDVDAIVPQSFLKDDSIDNKVLTRSDKNRGKSDNVPSEEVVKKMKNY
WRQLLNAKLITQRKFDNLTKAERGGLSELDKAGFIKRQLVETRQITKHV
AQILDSRMNTKYDENDKLIREVKVITLKSKLVSDFRKDFQFYKVREINN
YHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGR
DFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQIS
EFSKRVILADANLDKVLSAYNKHRDKPIREQAENIIHLFTLTNLGAPAA
FKYFDTTIDRKRYTSTKEVLDATLIHQSITGLYETRIDLSQLGGD

In some embodiments, the dCas9 is identical or substantially (e.g., at least 70, 75, 80, 85, 90, 95%) identical to SEQ ID NO:1.

As indicated above, the second Cas9 polypeptide can be a catalytically active or dead (catalytically inactive) Cas9 polypeptide. Exemplary dCas9 polypeptides are described above. Exemplary active Cas9 polypeptides can comprise sequences, for example, substantially (e.g., at least 70, 75, 80, 85, 90, 95%) identical to Cas9 from Streptococcus pyogenes (NCBI Reference Sequence: NC 017053.1, SEQ ID NO:2); Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisl (NCBI Ref: NC_018721.1); Streptococcus thermophilus (NCBI Ref: YP_820832.1), Listeria innocua (NCBI Ref: NP_472073.1), Campylobacter jejuni (NCBI Ref: YP_002344900.1) or Neisseria meningitidis (NCBI Ref: YP_002342100.1)

An exemplary active Cas9 polypeptide sequence from Streptococcus pyogenes is:

(SEQ ID NO: 2)
MDKKYSIGLAIGTNSVGWAVITDEYKVPSKKFKVLGNTDRHSIKKNLI
GALLFDSGETAEATRLKRTARRRYTRRKNRICYLQEIFSNEMAKVDDS
FFHRLEESFLVEEDKKHERHPIFGNIVDEVAYHEKYPTIYHLRKKLVD
STDKADLRLIYLALAHMIKFRGHFLIEGDLNPDNSDVDKLFIQLVQTY
NQLFEENPINASGVDAKAILSARLSKSRRLENLIAQLPGEKKNGLFGN
LIALSLGLTPNFKSNFDLAEDAKLQLSKDTYDDDLDNLLAQIGDQYAD
LFLAAKNLSDAILLSDILRVNTEITKAPLSASMIKRYDEHHQDLTLLK
ALVRQQLPEKYKEIFFDQSKNGYAGYIDGGASQEEFYKFIKPILEKMD
GTEELLVKLNREDLLRKQRTFDNGSIPHQIHLGELHAILRRQEDFYPF
LKDNREKIEKILTFRIPYYVGPLARGNSRFAWMTRKSEETITPWNFEE
VVDKGASAQSFIERMTNFDKNLPNEKVLPKHSLLYEYFTVYNELTKVK
YVTEGMRKPAFLSGEQKKAIVDLLFKTNRKVTVKQLKEDYFKKIECFD
SVEISGVEDRFNASLGTYHDLLKIIKDKDELDNEENEDILEDIVLTLT
LFEDREMIEERLKTYAHLFDDKVMKQLKRRRYTGWGRLSRKLINGIRD
KQSGKTILDFLKSDGFANRNFMQLIHDDSLTFKEDIQKAQVSGQGDSL
HEHIANLAGSPAIKKGILQTVKVVDELVKVMGRHKPENIVIEMARENQ
TTQKGQKNSRERMKRIEEGIKELGSQILKEHPVENTQLQNEKLYLYYL
QNGRDMYVDQELDINRLSDYDVDHIVPQSFLKDDSIDNKVLTRSDKNR
GKSDNVPSEEVVKKMKNYWRQLLNAKLITQRKFDNLTKAERGGLSELD
KAGFIKRQLVETRQITKHVAQILDSRMNTKYDENDKLIREVKVITLKS
KLVSDFRKDFQFYKVREINNYHHAHDAYLNAVVGTALIKKYPKLESEF
VYGDYKVYDVRKMIAKSEQEIGKATAKYFFYSNIMNFFKTEITLANGE
IRKRPLIETNGETGEIVWDKGRDFATVRKVLSMPQVNIVKKTEVQTGG
FSKESILPKRNSDKLIARKKDWDPKKYGGFDSPTVAYSVLVVAKVEKG
KSKKLKSVKELLGITIMERSSFEKNPIDFLEAKGYKEVKKDLIIKLPK
YSLFELENGRKRMLASAGELQKGNELALPSKYVNFLYLASHYEKLKGS
PEDNEQKQLFVEQHKHYLDEIIEQISEFSKRVILADANLDKVLSAYNK
HRDKPIREQAENIIHLFTLTNLGAPAAFKYFDTTIDRKRYTSTKEVLD
ATLIHQSITGLYETRIDLSQLGGD

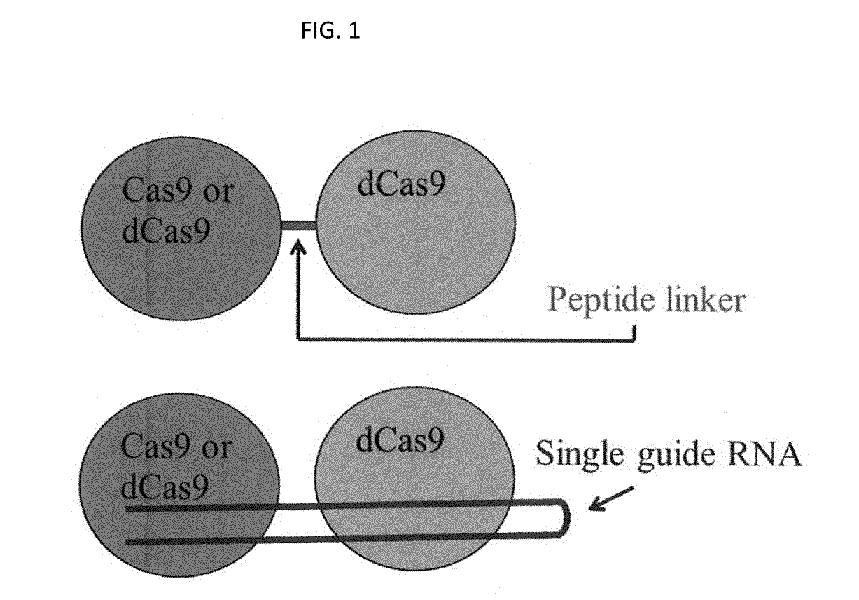

The polypeptide constructs comprise the first sequence-specific DNA-binding polypeptide linked to the second sequence-specific DNA-binding polypeptide. The two polypeptides can be linked as desired. In some embodiments, the two polypeptides are linked by a peptide bond (e.g., as a translational fusion), which can be fused directly or via a peptide linker. See, e.g., the top portion of FIG. 1. Exemplary peptide linker sequences contain Gly, Ser, Val and Thr residues. In some embodiments, other near neutral amino acids, such as Ala can also be used in the linker sequence. Amino acid sequences that may be usefully employed as linkers include those disclosed in Maratea et al. (1985) Gene 40:39-46; Murphy et al. (1986) Proc. Natl. Acad. Sci. USA 83:8258-8262; U.S. Pat. Nos. 4,935,233 and 4,751,180, each of which is hereby incorporated by reference in its entirety for all purposes and in particular for all teachings related to linkers. In some embodiments, the linker sequence may generally be from 1 to about 50 amino acids in length, e.g., 3, 4, 6, or 10 amino acids in length, but can be up to 100 or 200 amino acids in length in some embodiments.

The order of the two polypeptides in the fusion can vary. Thus, in some embodiments, the first sequence-specific DNA-binding polypeptide carboxyl terminus is linked directly or indirectly to the second sequence-specific DNA-binding polypeptide. In some embodiments, the second sequence-specific DNA-binding polypeptide carboxyl terminus is linked directly or indirectly to the first sequence-specific DNA-binding polypeptide.

In some embodiments, the first sequence-specific DNA binding protein is Cas9 (dCas9 or active Cas9 for example as described above) and the second sequence-specific binding protein an Ago protein, a transcription activator like effector nuclease (TALE) DNA binding domain, zinc finger.

Exemplary Ago polypeptides include those described in, e.g., Mallory, The Plant Cell 22(12):3879-3889 (2010). For example, the Ago protein can be a human, Arabidopsis, yeast, or other Ago protein or a substantially identical variant thereof that retains the ability to be a sequences-specific DNA binding protein.

An exemplary Ago protein from A. aeolicus (Yuan, et al. Mol. Cell 2005, 19:405-419) is provided as SEQ ID NO:3:

MGKEALLNLYRIEYRPKDTTFTVFKPTHEIQKEKLNKVRWRVFLQTGLP
TFRREDEFWCAGKVEKDTLYLTLSNGEIVELKRVGEEEFRGFQNERECQ
ELFRDFLTKTKVKDKFISDFYKKFRDKITVQGKNRKIALIPEVNEKVLK
SEEGYFLLHLDLKFRIQPFETLQTLLERNDFNPKRIRVKPIGIDFVGRV
QDVFKAKEKGEEFFRLCXERSTHKSSKKAWEELLKNRELREKAFLVVLE
KGYTYPATILKPVLTYENLEDEERNEVADIVRXEPGKRLNLIRYILRRY
VKALRDYGWYISPEEERAKGKLNFKDTVLDAKGKNTKVITNLRKFLELC
RPFVKKDVLSVEIISVSVYKKLEWRKEEFLKELINFLKNKGIKLKIKGK
SLILAQTREEAKEKLIPVINKIKDVDLVIVFLEEYPKVDPYKSFLLYDF
VKRELLKKXIPSQVILNRTLKNENLKFVLLNVAEQVLAKTGNIPYKLKE
IEGKVDAFVGIDISRITRDGKTVNAVAFTKIFNSKGELVRYYLTSYPAF
GEKLTEKAIGDVFSLLEKLGFKKGSKIVVHRDGRLYRDEVAAFKKYGEL
YGYSLELLEIIKRNNPRFFSNEKFIKGYFYKLSEDSVILATYNQVYEGT
HQPIKVRKVYGELPVEVLCSQILSLTLXNYSSFQPIKLPATVHYSDKIT
KLXLRGIEPIKKEGDIXYWL.

Naturally-occurring transcription activator like effectors (TALEs) are proteins secreted by Xanthomonas bacteria. The DNA binding domain of TALEs contains a highly conserved 33-34 amino acid sequence with the exception of the 12th and 13th amino acids. These two locations are highly variable (Repeat Variable Diresidue (RVD)) and show a strong correlation with specific nucleotide recognition. This simple relationship between amino acid sequence and DNA recognition has allowed for the engineering of specific DNA binding domains by selecting a combination of repeat segments containing the appropriate RVDs. See, e.g., US Patent Publication No. 2014/0256798, and U.S. Pat. Nos. 8,450,471; 8,440,431; and 8,440,432.

An exemplary TALE protein from Xanthomonas campestris pv. Armoraciae is provided below as SEQ ID NO:4:

MQWSGARALEALLTVAGELRGPPLQLDTGQLLKIAKRGGVTAVEAVHAW
RNALTGAPLNLTPEQVVAIASHDGGKQALETVQRLLPVLCQAHGLTPQQ
VVAIASHDGGKQALETVQRLLPVLCQAHGLTPEQVVAIASHDGGKQALE
TVQALLPVLCQAHGLTPEQVVAIASNGGGKQALETVQRLLPVLCQAHGL
TPQQVVAIASNGGGKQALETVQRLLPVLCQAHGLTPQQVVAIASNGGGK
QALETVQRLLPVLCQAHGLTPQQVVAIASNNGGKQALETVQRLLPVLCQ
AHGLTPQQVVAIASNGGGKQALETVQRLLPVLCQAHGLTPQQVVAIASH
DGGKQALETVQRLLPVLCQAHGLTPEQVVAIASNGGGKQALETVQRLLP
VLCQAHGLTPEQVVAIASHDGGKQALETVQRLLPVLCQAHGLTPQQVVA
IASNGGGRPALESIVAQLSRPDPALAALTNDHLVALACLGGRPALDAVK
KLE

Zinc fingers are proteins that fold based on the presence of a Zc atom and that bind DNA in a sequence-specific manner. Exemplary zinc fingers can be categorized into different structures, including but not limited to, e.g., Cys₂His₂, Gag-knuckle, Treble-clef, Zinc ribbon, Zn₂/Cys₆ zinc finger proteins.

An exemplary zinc finger protein is Zif268, provided below as SEQ ID NO:5:

MERPYACPVESCDRRFSRSDELTRHIRIHTGQKPFQCRICMRNFSRSDH
LTTHIRTHTGEKPFACDICGRKFARSDERKRHTKIHLRQKD

In other embodiments, the first sequence-specific DNA binding protein is an Ago protein. In these aspects, the second sequence-specific DNA binding protein can be, for example, a TALE DNA-binding domain, zinc finger, Cas9 (dCas9 or active) or a second Ago protein.

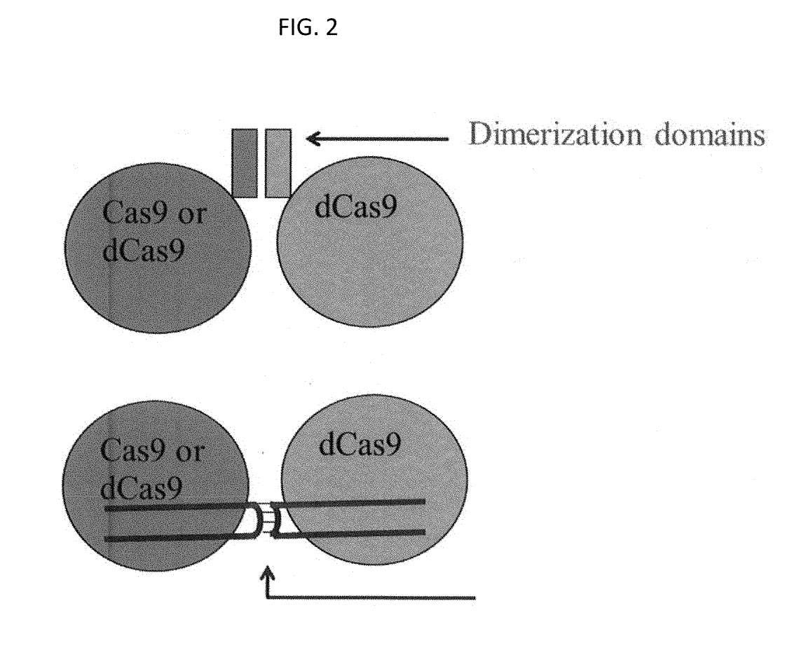

In some embodiments, rather than being linked by a peptide bond, the first sequence-specific DNA binding protein and the second sequence-specific DNA binding protein are linked via a dimerization domain, i.e., where the first sequence-specific DNA binding protein is fused to a first a dimerization domain and the second sequence-specific DNA binding protein is linked to a second dimerization domain such that the first and second dimerization domains link the two polypeptides via a covalent or non-covalent interaction between the first and second dimerization domains. See, e.g., the bottom portion of FIG. 2. Any convenient set of dimerization domains may be employed. The first and second dimerization domains may be homodimeric, such that they are made up of the same sequence of amino acids, or heterodimeric, such that they are made up of differing sequences of amino acids. Dimerization domains may vary, where domains of interest include, but are not limited to: ligands of target biomolecules, such as ligands that specifically bind to particular proteins of interest (e.g., protein:protein interaction domains), such as SH2 domains, Paz domains, RING domains, transcriptional activator domains, DNA binding domains, enzyme catalytic domains, enzyme regulatory domains, enzyme subunits, etc.

Exemplary dimerization domains include, but are not limited to, protein domains of the iDimerize inducible homodimer (e.g., DmrB) and heterodimer systems (e.g., DmrA and DmrC) and the iDimerize reverse dimerization system (e.g., DmrD) (Clackson et al. (1998) Proc. Natl. Acad. Sci. USA 95(18): 10437-10442; Crabtree, G. R. & Schreiber, S. L. (1996) Trends Biochem. Sci. 21(11): 418-422; Jin et al. (2000) Nat. Genet. 26(1): 64-66; Castellano et al. (1999) Curr. Biol. 9(7): 351-360; Crabtree et al. (1997) Embo. J. 16(18): 5618-5628; Muthuswamy et al. (1999) Mol. Cell. Biol. 19(10): 6845-6857).

In some embodiments, the dimerization domains are transcription activation domains. Transcription activation domains of interest include, but are not limited to: Group H nuclear receptor member transcription activation domains, steroid/thyroid hormone nuclear receptor transcription activation domains, synthetic or chimeric transcription activation domains, polyglutamine transcription activation domains, basic or acidic amino acid transcription activation domains, a VP16 transcription activation domain, a GAL4 transcription activation domains, an NF-.kappa.B transcription activation domain, a BP64 transcription activation domain, a B42 acidic transcription activation domain (B42AD), a p65 transcription activation domain (p65AD), or an analog, combination, or modification thereof.

In yet other embodiments, the first sequence-specific DNA binding protein that bind guide RNAs (e.g., Cas9 or Ago proteins) and the second sequence-specific DNA binding protein that bind guide RNAs (e.g., Cas9 or Ago proteins) are linked via separate guide RNAs that interact (e.g., hybridize) with each other. See, e.g., the top portion of FIG. 2. In these embodiments, the first sequence-specific DNA binding protein binds to a first guide RNA and the second sequence-specific DNA binding protein binds to a second guide RNA and the first and second guide RNAs include regions that interact (e.g., hybridize) with each other sufficiently such that the first sequence-specific DNA binding protein polypeptide and the second sequence-specific DNA binding protein are linked.

In yet other embodiments, the first sequence-specific DNA binding protein that bind guide RNAs (e.g., Cas9 or Ago proteins) and the second sequence-specific DNA binding protein that bind guide RNAs (e.g., Cas9 or Ago proteins) are linked via a single extended guide RNA that is bound by both of the first sequence-specific DNA binding protein and the second sequence-specific DNA binding protein. See, e.g., the bottom portion of FIG. 1. In some embodiments, the first and/or second sequence-specific DNA binding protein is a Cas9 protein (dCas9 or active Cas9). In these aspects, the guide RNA will comprise a first guide sequence and first trans-activating cr (tracr) sequence for the first dCas9 polypeptide and a second guide sequence and second tracr sequence for the second Cas9 polypeptide. In some embodiments, for example, the RNA will comprise from 5′ to 3′ as follows: a first guide sequence, first tracr sequence, second guide sequence and second tracr sequence. In some embodiments, for example, the RNA will comprise from 5′ to 3′ as follows: a first guide sequence, second guide sequence, second tracr sequence, and first tracr sequence. This latter embodiment is depicted in the bottom of FIG. 1.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within, or targeting to, the target sequence. Similarly, cleavage of a target polynucleotide sequence may be evaluated in vitro by providing the target sequence, components of a CRISPR complex, including the guide sequence to be tested and a control guide sequence different from the test guide sequence, and comparing binding or rate of cleavage at the target sequence between the test and control guide sequence reactions.

A guide sequence may be selected to target any target sequence. In some embodiments, the target sequence is a sequence within a genome of a cell. Exemplary target sequences include those that are unique in the target genome.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106(1): 23-24; and PA Carr and GM Church, 2009, Nature Biotechnology 27(12): 1151-62).

Any RNA sequence that mimics the tracrRNA sequence (i.e., the stem loop recognized by the Cas9 polypeptide) or any RNA sequences that function as a tracrRNA are “tracr sequences.” Exemplary loop forming sequences for use in hairpin structures are four nucleotides in length, and in some embodiments have the sequence GAAA. However, longer or shorter loop sequences may be used, as may alternative sequences. The sequences can include a nucleotide triplet (for example, AAA), and an additional nucleotide (for example C or G). Examples of loop forming sequences include CAAA and AAAG. In an embodiment, the transcript or transcribed polynucleotide sequence has at least two or more hairpins. In some embodiments, the transcript has two, three, four or five hairpins. In a further embodiment, the transcript has at most five hairpins. In some embodiments, the single transcript further includes a transcription termination sequence; e.g., a polyT sequence, for example having six T nucleotides. See, e.g., U.S. Pat. No. 8,697,359.

Also provided is a cell comprising a polypeptide construct comprising the first sequence-specific DNA binding protein linked to the second sequence-specific DNA binding protein. In some embodiments, the cell comprises polynucleotides encoding the polypeptide construct comprising the first sequence-specific DNA binding protein linked directly or indirectly (i.e., via a linker) as a translational fusion to the second sequence-specific DNA binding protein. Exemplary cells can include, but are not limited to prokaryotic cells (e.g., E. coli), animal cells, fungal cells or plant cells. Exemplary animal cells include but are not limited to mammalian cells, e.g., mouse, rat, or human cells. In some embodiments, the polynucleotide further includes a promoter controlling expression of the polypeptide construct. Exemplary promoters can be constitutive, inducible, or cell-type or tissue-specific.

Also provided are nucleic acids (e.g., isolated DNA or RNA) encoding the polypeptide constructs. In some embodiments, the nucleic acids have been codon-optimized for expression in a cell, including but not limited, to the cells listed above. In some embodiments, the nuclei acids further comprising coding and expression sequences for expression of one or more guide RNA.

In some embodiments, the nucleic acids further comprise one or more nuclear localization signal sequence (NLS) translationally-fused to the protein construct. Non-limiting examples of NLSs include an NLS sequence derived from: the NLS of the SV40 virus large T-antigen, having the amino acid sequence PKKKRKV (SEQ ID NO:6); the NLS from nucleoplasmin (e.g. the nucleoplasmin bipartite NLS with the sequence KRPAATKKAGQAKKKK (SEQ ID NO: 7)); the c-myc NLS having the amino acid sequence PAAKRVKLD (SEQ ID NO: 8) or RQRRNELKRSP (SEQ ID NO: 9); the hRNPAI M9 NLS having the sequence NQSSNFGPMKGGNFGGRSSGPYGGGGQYFAKPRNQGGY (SEQ ID NO: 10); the sequence RMRIZFKNKGKDTAELRRRRVEVSVELRKAKKDEQILKRRNV (SEQ ID NO: 11) of the IBB domain from importin-alpha; the sequences VSRKRPRP (SEQ ID NO: 12) and PPKKARED (SEQ ID NO: 13) of the myoma T protein; the sequence PQPKKKPL (SEQ ID NO: 14) of human p53; the sequence SALIKKKKKMAP (SEQ ID NO: 15) of mouse c-abl IV; the sequences DRLRR (SEQ ID NO: 16) and PKQKKRK (SEQ ID NO: 17) of the influenza virus NS1; the sequence RKLKKKIKKL (SEQ ID NO: 18) of the Hepatitis virus delta antigen; the sequence REKKKFLKRR (SEQ ID NO: 19) of the mouse Mx protein; the sequence KRKGDEVDGVDEVAKKKSKK (SEQ ID NO: 20) of the human poly(ADP-ribose) polymerase; and the sequence RKCLQAGMNLEARKTKK (SEQ ID NO: 21) of the steroid hormone receptors (human) glucocorticoid.

The protein constructs can be introduced into a cell by expression of a nucleic acid encoding the protein construct, wherein the nucleic acid is in the cell, or the protein construction can be introduced directly into the cell. Transformation of cells with nucleic acids, as well as a variety of expression cassettes and expression vectors are known. Basic texts disclosing the general methods of introducing nucleic acids into cells and recombinant techniques include Sambrook and Russell, Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994-1999).

In some embodiments, the protein construct, with or without the corresponding guide RNA, can be introduced into the cell. In some of these embodiments, the protein constructs comprise an NLS, e.g., as described above. Introduction of protein constructs into cells can be performed, for example, by injection, electroporation, lipid delivery (e.g., US Patent Publication no. 20150071903), or mixture with polyarginine (e.g., Kanwar, et al., Anticancer Drugs 23(5):471-82 (2012)).

Also provided are RNA molecules comprising a first guide sequence for the first dCas polypeptide and a second guide RNA for the second Cas9 polypeptide, for example as described above. Such RNA molecules will generally comprise two tracr sequences, one for the first dCas9 polypeptide to bind and a second tracr sequence for the second Cas9 polypeptide to bind. In some embodiments, for example, the RNA will comprise from 5′ to 3′ as follows: a first guide sequence, first tracr sequence, second guide sequence and second tracr sequence. In some embodiments, for example, the RNA will comprise from 5′ to 3′ as follows: a first guide sequence, second guide sequence, second tracr sequence, and first tracr sequence.

Also provided are expression cassettes that encode the RNA molecules described above. In some embodiments, a nucleic acid will comprise such expression cassettes as well as an expression cassette encoding a polypeptide construct as described above.

In the claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded.

The above disclosure is provided to illustrate the invention but not to limit its scope. Variants of the invention will be readily apparent to one of ordinary skill in the art and are encompassed by the appended claims. All publications, databases, internet sources, patents, patent applications, and accession numbers cited herein are hereby incorporated by reference in their entireties for all purposes. To the extent such documents incorporated by reference contradict the disclosure contained in the specification, the specification is intended to supersede and/or take precedence over any contradictory material.

1. A polypeptide construct comprising a first sequence-specific DNA binding protein, selected from a Cas9 protein or an Ago protein, linked to a second sequence-specific DNA binding protein.

2. The polypeptide construct of claim 1, wherein the first sequence-specific DNA binding protein is a Cas 9 protein.

3. The polypeptide construct of claim 2, wherein the Cas9 protein is a dCas9 protein

4. The polypeptide construct of claim 2, wherein the Cas9 protein is an active Cas9 protein.

5. The polypeptide construct of claim 2, wherein a first dCas9 polypeptide linked to a second Cas9 polypeptide.

6. The polypeptide construct of claim 5, wherein the second Cas9 polypeptide is a second dCas9 polypeptide.

7. The polypeptide construct of claim 5, wherein the second Cas9 polypeptide is a nuclease-active Cas9 polypeptide.

8. The polypeptide construct of claim 1, wherein the first sequence-specific DNA binding protein is covalently linked to the second sequence-specific DNA binding protein.

9. The polypeptide construct of claim 1, wherein the first sequence-specific DNA binding protein is covalently linked to the second sequence-specific DNA binding protein as a translational fusion protein.

10. The polypeptide construct of claim 1, wherein the first sequence-specific DNA binding protein is linked to the second sequence-specific DNA binding protein via a dimerization domain(s).

11. The polypeptide construct of claim 5, wherein the first dCas9 polypeptide is bound to a first guide RNA and the second Cas9 polypeptide is bound to a second guide RNA and the first dCas9 polypeptide is linked to the second Cas9 polypeptide via an interaction of the first guide RNA to the second guide RNA.

12. The polypeptide construct of claim 5, wherein the first dCas9 polypeptide and the second Cas9 polypeptide are bound to an RNA, said RNA comprising
a first Cas9 guide sequence; a first tracr RNA sequence; a second Cas9 guide sequence; and a second tracr RNA sequence, wherein:
the first dCas polypeptide binds to the first tracr RNA sequence and the second Cas9 polypeptide binds to the second tracr sequence; or
the first dCas polypeptide binds to the second tracr RNA sequence and the second Cas9 polypeptide binds to the first tracr sequence,
such that the first dCas polypeptide and the second Cas polypeptide are bound together via the RNA.

13. The polypeptide construct of claim 1, wherein the second sequence-specific DNA binding protein comprises a TALE DNA binding domain or a zinc finger protein.

14. The polypeptide construct of claim 1, wherein the first sequence-specific DNA binding protein is an Ago protein.

15. The polypeptide construct of claim 14, wherein the second sequence-specific DNA binding protein comprises a Cas9 protein, an Ago protein, a TALE DNA-binding domain, or a zinc finger protein.

16. A cell comprising the polypeptide construct of claim 1.

17. The cell of claim 16, wherein the cell is a eukaryotic cell.

18. The cell of claim 17, wherein the cell is a human cell.

19. A nucleic acid encoding the translational fusion protein of claim 9.


SEQUENCE LISTING (21 sequences)

SEQ ID NO: 1 - protein, 1368 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 2 - protein, 2736 aa, Streptococcus pyogenes
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDMDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 3 - protein, 706 aa, Aquifex pyrophilus
  misc_feature (214)..(214): Xaa can be any naturally occurring amino acid
  misc_feature (278)..(278): Xaa can be any naturally occurring amino acid
  misc_feature (450)..(450): Xaa can be any naturally occurring amino acid
  misc_feature (665)..(665): Xaa can be any naturally occurring amino acid
  misc_feature (689)..(689): Xaa can be any naturally occurring amino acid
  misc_feature (703)..(703): Xaa can be any naturally occurring amino acid
MGKEALLNLYRIEYRPKDTTFTVFKPTHEIQKEKLNKVRWRVFLQTGLPTFRREDEFWCAGKVEKDTLYLTLSNGEIVELKRVGEEEFRGFQNERECQELFRDFLTKTKVKDKFISDFYKKFRDKITVQGKNRKIALIPEVNEKVLKSEEGYFLLHLDLKFRIQPFETLQTLLERNDFNPKRIRVKPIGIDFVGRVQDVFKAKEKGEEFFRLCXERSTHKSSKKAWEELLKNRELREKAFLVVLEKGYTYPATILKPVLTYENLEDEERNEVADIVRXEPGKRLNLIRYILRRYVKALRDYGWYISPEEERAKGKLNFKDTVLDAKGKNTKVITNLRKFLELCRPFVKKDVLSVEIISVSVYKKLEWRKEEFLKELINFLKNKGIKLKIKGKSLILAQTREEAKEKLIPVINKIKDVDLVIVFLEEYPKVDPYKSFLLYDFVKRELLKKXIPSQVILNRTLKNENLKFVLLNVAEQVLAKTGNIPYKLKEIEGKVDAFVGIDISRITRDGKTVNAVAFTKIFNSKGELVRYYLTSYPAFGEKLTEKAIGDVFSLLEKLGFKKGSKIVVHRDGRLYRDEVAAFKKYGELYGYSLELLEIIKRNNPRFFSNEKFIKGYFYKLSEDSVILATYNQVYEGTHQPIKVRKVYGELPVEVLCSQILSLTLXNYSSFQPIKLPATVHYSDKITKLXLRGIEPIKKEGDIXYWL

SEQ ID NO: 4 - protein, 493 aa, Xanthomonas campestris pv. Armoraciae
MQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASHDGGKQALETVQRLLPVLCQAHGLTPEQVVAIASHDGGKQALETVQALLPVLCQAHGLTPEQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASHDGGKQALETVQRLLPVLCQAHGLTPEQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKLE

SEQ ID NO: 5 - protein, 90 aa, Artificial Sequence
  synthetic zinc finger protein Zif268
MERPYACPVESCDRRFSRSDELTRHIRIHTGQKPFQCRICMRNFSRSDHLTTHIRTHTGEKPFACDICGRKFARSDERKRHTKIHLRQKD

SEQ ID NO: 6 - protein, 7 aa, Artificial Sequence
  synthetic nuclear localization signal sequence (NLS) of the SV40 virus large T-antigen
PKKKRKV

SEQ ID NO: 7 - protein, 16 aa, Artificial Sequence
  synthetic nuclear localization signal sequence (NLS) from nucleoplasmin
KRPAATKKAGQAKKKK

SEQ ID NO: 8 - protein, 9 aa, Artificial Sequence
  synthethic c-myc nuclear localization signal sequence (NLS)
PAAKRVKLD

SEQ ID NO: 9 - protein, 11 aa, Artificial Sequence
  synthethic c-myc nuclear localization signal sequence (NLS)
RQRRNELKRSP

SEQ ID NO: 10 - protein, 38 aa, Artificial Sequence
  synthetic hRNPAI M9 nuclear localization signal sequence (NLS)
NQSSNFGPMKGGNFGGRSSGPYGGGGQYFAKPRNQGGY

SEQ ID NO: 11 - protein, 42 aa, Artificial Sequence
  synthetic nuclear localization signal sequence (NLS) of the IBB domain from importin-alpha
RMRIZFKNKGKDTAELRRRRVEVSVELRKAKKDEQILKRRNV

SEQ ID NO: 12 - protein, 8 aa, Artificial Sequence
  synthetic nuclear localization signal sequence (NLS) of the myoma T protein
VSRKRPRP

SEQ ID NO: 13 - protein, 8 aa, Artificial Sequence
  synthetic nuclear localization signal sequence (NLS) of the myoma T protein
PPKKARED

SEQ ID NO: 14 - protein, 8 aa, Artificial Sequence
  synthetic nuclear localization signal sequence (NLS) of human p53
PQPKKKPL

SEQ ID NO: 15 - protein, 12 aa, Artificial Sequence
  synthetic nuclear localization signal sequence (NLS) of mouse c-abl IV
SALIKKKKKMAP

SEQ ID NO: 16 - protein, 5 aa, Artificial Sequence
  synthetic nuclear localization signal sequence (NLS) of the influenza virus NS1
DRLRR

SEQ ID NO: 17 - protein, 7 aa, Artificial Sequence
  synthetic nuclear localization signal sequence (NLS) of the influenza virus NS1
PKQKKRK

SEQ ID NO: 18 - protein, 10 aa, Artificial Sequence
  synthetic nuclear localization signal sequence (NLS) of the Hepatitis virus delta antigen
RKLKKKIKKL

SEQ ID NO: 19 - protein, 10 aa, Artificial Sequence
  synthetic nuclear localization signal sequence (NLS) of the mouse Mx protein
REKKKFLKRR

SEQ ID NO: 20 - protein, 20 aa, Artificial Sequence
  synthetic nuclear localization signal sequence (NLS) of the human poly(ADP-ribose) polymerase
KRKGDEVDGVDEVAKKKSKK

SEQ ID NO: 21 - protein, 17 aa, Artificial Sequence
  synthetic nuclear localization signal sequence (NLS) of the steroid hormone receptors (human) glucocorticoid
RKCLQAGMNLEARKTKK